(19) United States
(12) Patent Application Publication
(10) Pub. No.: US 2007/0141118 A1
(43) Pub. Date: Jun. 21, 2007

(54) Layered dosage form for a medicated tampon assembly

(76) Inventors: Joyce A. Damico, Neenah, WI (US); Bruce M. Siebers, Kimberly, WI (US); Patrick R. Hastings, Combined Locks, WI (US); Steven R. Burnett, West Chester, OH (US); Chris Clendening, Cleves, OH (US)

Correspondence Address: KIMBERLY-CLARK WORLDWIDE, INC., 401 NORTH LAKE STREET, NEENAH, WI, 54956, US

(21) Appl. No.: 11/303,802

(22) Filed: Dec. 15, 2005

(51) Int. Cl. A61K 31/554; A61K 31/4152; A61K 31/365

(52) U.S. Cl. 424/431; 424/443; 514/211.07; 514/420; 514/406; 514/536; 514/471; 514/569

(57) ABSTRACT

A tampon adapted to deliver a therapeutic agent includes a tampon body having a distal end and a dosage form affixed to the distal end of the tampon body, the dosage form including a first active layer dispersible by a first mechanism, and an attachment layer dispersible by a second mechanism. Mechanisms include melting and dissolution.

### BACKGROUND

Aspects of the present invention pertain to methods of manufacturing medicated tampon assemblies used for the application of various therapeutic treatments or preparations into the vaginal or other cavity.

Many disease states and physiological conditions may occur in a woman, including symptoms associated with premenstrual syndrome, menstruation, and menopause. These symptoms may include dysmenorrhea (menstrual cramping), irritability, water retention, moodiness, depression, anxiety, skin changes, headaches, breast tenderness, tension, weight gain, cravings, fatigue hot flashes, itching, and other associated sensory maladies. Many of these symptoms are due to changes in hormonal levels throughout the menstrual cycle. One example that affects a large number of post-pubescent women is dysmenorrhea, which is the occurrence of painful uterine cramps during menstruation. Menstrual cramping is associated with increased levels of prostaglandin F2α, prostaglandin E2, and, in some cases, leukotrienes in the endometrium and menstrual fluid. These eicosinoids lead to restricted blood flow to the uterus and increased uterine contractions, causing pain.

Various analgesics may be effective in limiting the pain from dysmenorrhea; however some orally-delivered analgesics can cause nausea and vomiting or other untoward side effects; therefore alternative routes of analgesic delivery are of interest.

Attempts have been made to deliver analgesics in the vicinity of the cervix and the vaginal mucosa using various vaginally-inserted devices and methods. Because many of these symptoms typically occur in conjunction with menstruation, some have tried to combine an analgesic with a tampon by coating the tampon, dipping the tampon, or by combining the analgesic with the tampon materials.

For example, in a method of preparation of such a product appropriate for a laboratory setting, a formulation of a fatty compound excipient and an analgesic are heated to a liquid state. Constant mixing of the heated formulation is required to produce a homogeneous formulation. The formulation is then poured onto the tip of a tampon held in a form to contain the liquid. As the formulation cools, the ingredients solidify into a solid waxy substance that has adhered to the absorbent material of the tampon and is thereby securely fastened to the tip of the tampon.

### SUMMARY

Several problems are inherent in a process that attempts to introduce a formulation including a therapeutic agent into or onto a tampon by coating, dipping, solidifying, or the like. Processes such as these can result in a formulation including a therapeutic agent absorbing into the tampon during manufacture or use, thus preventing the therapeutic agent from being delivered to the user. In addition, different styles and sizes of tampons may have different densities and will absorb an applied liquid formulation including a therapeutic agent differently, resulting in variability in therapeutic agent concentrations across such different tampons.

These problems are solved by various aspects of the present invention including a medicated tampon assembly having a dosage form in a layered construction. Each layer of the dosage form disperses by a different mechanism, or by the same but differentiated mechanism. For example, one layer may have a different melt temperature and/or heat of fusion than an adjacent layer. In this example, the melt temperatures/heats of fusion may increase moving toward the tampon. In another example, a first layer may disperse by melting, and an adjacent layer may disperse by dissolution. A therapeutic agent may be contained in the layer with the lowest melt temperature/heat of fusion or the most immediately-dispersible mechanism. Such layer is generally positioned furthest from the tampon. During the initial minutes of use, the layer including the therapeutic agent disperses first and the layer closest to the tampon disperses last. The progressive dispersal of layers forms a shield that minimizes absorption of the therapeutic agent into the tampon. Such a construction allows for robust mechanical attachment of the dosage form to the tampon and minimizes absorption of the therapeutic agent into the tampon during manufacture and during the initial minutes of use.

More specifically, the present invention provides a tampon adapted to deliver a therapeutic agent, the tampon including a tampon body having a distal end and a dosage form affixed to the distal end of the tampon body, the dosage form including a first active layer dispersible by a first mechanism, the active layer including the therapeutic agent, and an attachment layer dispersible by a second mechanism, wherein the first mechanism is different from the second mechanism.

The present invention also provides a dosage form including a therapeutic agent, the dosage form including a first active layer dispersible by a first mechanism, the first active layer including the therapeutic agent, and an attachment layer dispersible by a second mechanism, wherein the first mechanism is different from the second mechanism, wherein the first active layer and the attachment layer are adapted such that the first active layer is substantially dispersed from the dosage form prior to the complete dispersal of the attachment layer.

Aspects of the present invention relate to a dosage form that is integral with or associated with a feminine care product. The dosage form including the therapeutic agent and excipients may include any therapeutic agent that may be absorbed into the body through the vaginal or other epithelium, or deposited topically on the vaginal or other epithelium, for the purposes of treating a physiological disease, state, or condition.

Other objects and advantages of aspects of the present invention will become more apparent to those skilled in the art in view of the following description and the accompanying drawings.

### BRIEF DESCRIPTION OF THE DRAWINGS

FIG. 1 is a schematic view of a two-piece tampon assembly to be used in conjunction with a medicated tampon assembly.

FIG. 2 is a cross-sectional schematic view of the tampon assembly illustrated in FIG. 1.

FIG. 3 is a cross-sectional elevation view of a dosage form manufactured for the medicated tampon assembly of FIG. 1.

### DETAILED DESCRIPTION OF THE PREFERRED EMBODIMENTS

Aspects of the present invention as described herein will be described for exemplary purposes using a tampon as an example of a feminine care product. Aspects of the present invention, however, apply equally to other forms of products, including tampon-like devices and vaginally-inserted devices, and should not be limited to the example described herein. In addition, although the example described includes a tampon with absorbent material, a product without absorbent material, such as a tampon applicator or other similar applicator, is also contemplated within the various aspects of the present invention. Also contemplated is the use of aspects of the present invention described herein in conjunction with non-catamenial feminine products such as incontinence products, including female incontinence inserts.

The term “surface” and its plural generally refer herein to the outer or the topmost boundary of an object.

The term “dosage form” is used herein as a generic term for a unit form including a formulation that includes a therapeutic agent. The dosage form includes a discrete and consistent quantity of the therapeutic agent to allow for consistent dosing of one receiving the dosage form. The dosage form may be a suppository, a capsule, or any other suitable form. The dosage form may also be spherical, ovoid, domal, generally flat, or any other suitable shape dictated by the needs of the application of the dosage form. The dosage form may have convex, concave, planar, arcuate, or any other suitable surfaces as dictated by the needs of the application of the dosage form.

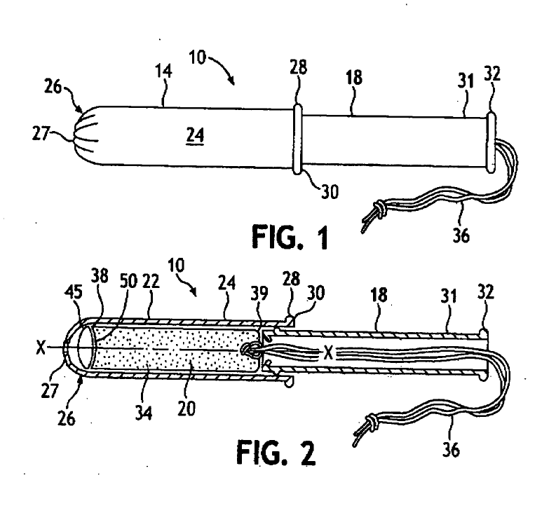

FIGS. 1-2 illustrate a delivery device in the form of a medicated tampon assembly 10, including a first member 14 and a second member 18, which is designed to house a tampon 20 and provide a comfortable means of inserting the tampon 20 into a woman's vagina.

The first member 14 of the medicated tampon assembly 10 may be in the form of a spirally wound, convolutely wound or longitudinally seamed hollow tube which is formed from paper, paperboard, cardboard, plastic, other suitable material, or a combination of such materials. Any plastic in the first member 14 is preferably polyethylene, but may be polypropylene or other suitable plastic. The first member 14, also commonly referred to as an outer tube, may be of any suitable dimensions necessary to house a particular size of tampon 20. The first member 14 has a wall 22 with an outside or exterior surface 24.

The first member 14 is sized and configured to house the tampon 20, and should have a substantially smooth exterior surface 24 which will facilitate insertion of the first member 14 into a woman's vagina. When the exterior surface 24 is smooth and/or slippery, the first member 14 will easily slide into a woman's vagina without subjecting the internal tissues of the vagina to abrasion. The first member 14 may be coated to give it a high slip characteristic. Wax, polyethylene, a combination of wax and polyethylene, cellophane and clay are representative coatings that may be applied to the first member 14 to facilitate comfortable insertion. The first member 14 itself may be formulated to give it a high slip characteristic, including the addition of additives to the resin from which the first member is made, or by an alteration in physical structure of the exterior surface 24, such as adding pebbling or other bumps, to decrease the amount of surface area in contact with the vaginal or other epithelium.

Referring to FIG. 1, an insertion tip 26 is shown having a plurality petals 27 that may radially open such that the insertion tip 26 has a diameter approximately equal to or greater than the diameter of the first member 14. The petals 27 may be either even or odd in number and may be equally spaced apart or non-uniformly arranged. In another aspect, the insertion tip 26 may be provided without petals 27.

The first member 14 may have a fingergrip ring 28 located proximate a first member receiving end 30. The fingergrip ring 28 provides an enlarged surface onto which one or more fingers of the user may rest.

As stated above, the medicated tampon assembly 10 includes a second member 18, also commonly referred to as an inner tube. The second member 18, like the first member 14, may be a spirally wound, a convolutely wound or a longitudinally seamed hollow tube constructed from paper, paperboard, cardboard, plastic, other suitable material, or a combination thereof. The second member 18 may be constructed of the same material as the first member 14 or it may be made out of a different material. The second member 18 may also be a solid stick or use some other unique shape. It is also possible to form a finger flange 32 on the free end 31 of the second member 18 to provide an enlarged surface onto which the user's forefinger may rest. The finger flange 32 thereby functions as a seat for the forefinger and facilitates movement of the second member 18 into the first member 14.

In an alternate aspect of the present invention (not shown), the first member 14 and second member 18 together may be replaced by a stick applicator. The stick applicator is used to insert the tampon 20, after which the stick applicator is withdrawn.

A tampon 20 may be an absorbent member primarily designed to be worn by a woman during her menstrual period to absorb menses and other body fluids. The tampon 20 includes a tampon body 34 and a withdrawal string 36. The tampon body 34 is normally compressed into the form of a cylinder and may have a blunt, rounded or shaped forward end. The tampon body 34 has a forward or distal end 38 that is closer to the cervix when the tampon 20 is in use. The tampon body 34 also has a proximal end 39 that is closer to the vaginal opening when the tampon 20 is in use. The tampon 20 commonly has a withdrawal string 36 fastened to the tampon body 34 and extending from the proximal end 39. The withdrawal string 36 serves as a means for withdrawing the tampon 20 from the woman's vagina. Catamenial tampons suitable for use in aspects of the present invention include any absorbent material as is known in the art. The tampon body 34 may be formed into specific shapes such as various cup shapes to enhance the therapeutic agent contact area with the cervix, posterior fornix, anterior fornix, lateral fornices, vaginal epithelium areas, or conformance to other anatomical areas within the vaginal or other cavity.

The medicated tampon assembly 10 includes a dosage form 45 in addition to the tampon 20. The dosage form 45 may be combined with any absorbent tampon design. The dosage form 45 is preferably positioned at the distal end 38 of the tampon body 34. In alternate aspects of the present invention, the dosage form 45 may be positioned at the proximal or string end 39 of the tampon body 34, or any other suitable position between the distal and proximal ends 38, 39 of the tampon body 34. The dosage form 45 may be designed to partially or fully cover the distal end 38 of the tampon body 34. The tampon body 34 may be formed into specific shapes such as various cup shapes to enhance the therapeutic agent contact area with the cervix, posterior fornix, vaginal or other epithelium areas, or conformance to other anatomical areas within the vaginal or other cavity.

In other various aspects of the present invention, the tampon 20 may include a recess, a dimple, a depression, a concavity, or a reservoir (generically a recess) 50 at the distal end 38 (see FIG. 2), at the proximal end 39, or at any point between the distal and proximal ends 38, 39. The recess 50 is designed to accommodate the dosage form 45. The dosage form 45 may be applied to the recess 50 by any method described herein or by any other suitable method. In an alternate aspect of the present invention, the recess 50 may be formed as a simple dimple. In other alternate aspects of the present invention, the distal end 38 of the tampon 20 may flat, convex, or of any other suitable shape or arrangement.

In one aspect of the present invention, the dosage form 45 may be produced in any suitable form including, but not limited to, tablets, capsules, suppositories, gels, disks, lozenges, films, coatings, and other forms. In an alternate aspect of the present invention, the dosage form 45 may be produced in an encapsulated form.

Additionally, the dosage form 45 may be formed in any shape to promote attachment to the distal end 38 of the tampon body 34 and/or to promote contact with anatomical structures such as the vaginal epithelium, the posterior fornix, the anterior fornix, the lateral fornices, the cervix, or other structures. In another aspect of the present invention, the dosage form 45 may also be used independently of a tampon 20 to deliver a therapeutic agent to a vaginal environment, the vaginal mucosa, the cervix, or other suitable structure or environment.

The dosage form 45 of the medicated tampon assembly 10 includes a therapeutic agent. For the purposes of the aspects of the present invention, any therapeutic agent that will treat the vaginal or other cavity or will be absorbed into a user's body through the vaginal or other epithelium for the purposes of treating diseases or conditions, promoting the growth of normal vaginal flora, or promoting vaginal health may be used. Examples of therapeutic agents that may be used include, but are not limited to, botanicals, vitamins, moisturizers, antifungal agents, antibacterial agents, pro-biotic agents, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

Some therapeutic agents for use in aspects of the present invention are absorbable through the vaginal epithelium and travel to the uterus by a unique portal of veins and arteries that are known to exist between the vagina, the cervix, and the uterus. This anastomosis eliminates first-pass metabolism by the liver, effectively delivering higher concentrations of the therapeutic agent to the uterus than would otherwise be available via oral dosing. Those of skill in the art know the efficacy of various therapeutic agents when introduced at a particular anatomical location. For example, when the therapeutic agent is selected to treat dysmenorrhea, it preferably is selected from the following group: nonsteroidal anti-inflammatory drugs (NSAIDs), prostaglandin inhibitors, COX-2 inhibitors, local anesthetics, calcium channel blockers, potassium channel blockers, β-adrenergic agonists, leukotriene blocking agents, smooth muscle inhibitors, and drugs capable of inhibiting dyskinetic muscle contraction.

COX-2 inhibitors, such as Celecoxib, Meloxicam, Rofecoxib, and Flosulide are novel anti-inflammatory and analgesic compounds. These compounds effectively inhibit production of COX-2 (cyclooxygenase-2) enzyme that is induced by pro-inflammatory stimuli in migratory cells and inflamed tissue. Because COX-2 is also involved in reproductive processes, selective COX-2 inhibitors will reduce uterine contractions in pre-term labor and relieve painful uterine contractions associated with dysmenorrhea by blocking prostaglandin receptors in the uterus. Additionally, they may reduce endometrial bleeding.

Preferred NSAIDs include aspirin, ibuprofen, indomethacin, phenylbutazone, bromfenac, sulindac, nabumetone, ketorolac, mefenamic acid, and naproxen. Preferred local anesthetics include lidocaine, mepivacaine, etidocaine, bupivacaine, 2-chloroprocaine hydrochloride, procaine, and tetracaine hydrochloride. Preferred calcium channel antagonists include diltaizem, israpidine, nimodipine, felodipine, verapamil, nifedipine, nicardipine, and bepridil. Preferred potassium channel blockers include dofetilide, e-4031, imokalant, sematilide, ambasilide, azimilide, tedisamil, rp58866, sotalol, piroxicam, and ibutilide. Preferred β-adrenergic agonists include terbutaline, salbutamol, metaproterenol, and ritodrine. Vasodilators, which are believed to relieve muscle spasm in the uterine muscle, include nitroglycerin, isosorbide dinitrate, and isosorbide mononitrate.

Examples of beneficial botanicals may include, but are not limited to, Agnus castus, aloe vera, comfrey, calendula, dong quai, black cohosh, chamomile, evening primrose, Hypericum perforatum, licorice root, black currant seed oil, St. John's wort, tea extracts, lemon balm, capsicum, rosemary, Areca catechu, mung bean, borage seed oil, witch hazel, fenugreek, lavender, and soy. Vaccinium extracts commonly derived from many members of the heath family, cranberries such as blueberries, and azaleas (Rhododendron spp.) as well as from red onion skin and short and long red bell peppers, Beta vulgaris (beet) root extract, and capsanthin may also be used. Other berries that have applicability are whortleberry, lingenberry, chokeberry, sweet rowan, rowanberry, seabuckhrouberry, crowberry, strawberries, and gooseberries.

Other beneficial agents that may be used include, but are not limited to, vitamins, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

These beneficial agents promote epithelial health in the vaginal region. The idea is to modulate the vaginal environment to enhance the wellness of this anatomical region. These benefits can be rather simple, for example increasing comfort by providing moisturization and/or lubricity. These benefits can also be more complex, for example modulating epithelial cell function to address vaginal atrophy. The beneficial therapeutic agents may reduce negative sensations such as stinging, burning, itching, etc, or introduce positive sensations to improve comfort.

For example, many therapeutic benefits have been ascribed to a large number of different botanical preparations. Preparations may include water-in-oil emulsions, oil-in-water emulsions, gel, liquid, dispersion, powder, and anhydrous systems, ointment, or salve, such as a botanical oil in an anhydrous base (e.g., petrolatum), or polyethylene glycol based systems. Also, botanicals are often prepared or extracted under conditions to generate water-soluble or oil-soluble extracts. These extracts are usually compositionally different and may have different skin and vaginal health benefits. Processing conditions will have an effect on the type of formulation that can be used and this will restrict the type of botanical (water or oil type) selected. Therefore, wide ranges of botanicals have utility in various aspects of the present invention. Botanicals can possess a variety of actives and activities that can include, but are not necessarily limited to, analgesics, antimicrobials, pro-biotic agents, anti-inflammatory compounds, anti-virals, enzymes, enzyme inhibitors, enzyme substrates, enzyme cofactors, ions, ion chelators, lipids, lipid analogs, lipid precursors, hormones, inflammatory mediators, inflammatory agonists, oxidants, antioxidants, humectants, growth factors, sugars, oligosaccarides, polysaccarides, vasodilators, and potential combinations thereof. It is understood that, for the purposes of the various aspects of the present invention, the botanicals can be combined with any number of non-botanical active ingredients as well.

The dosage form 45 may include any therapeutic agent, excipient, formulation, compound, or combination thereof that is desirable to introduce into the vaginal or other cavity, including excipients to promote the functionality of the therapeutic agent. The excipients may assist the release of the therapeutic agent, or assist in the absorbency of the therapeutic agent into the vaginal or other epithelium. The use of excipients to facilitate the formulation, delivery, stability, and aesthetic properties of a therapeutic agent delivery system is well known to those familiar with the art.

In other aspects of the present invention, the dosage form 45 may be designed to melt at approximately body temperature, or to dissolve or otherwise disperse in the presence of a sufficient aqueous or other liquid trigger, or appropriate chemistry, such as a suitable pH. The dosage form 45 may also be provided with one or more coatings, such as a surfactant.

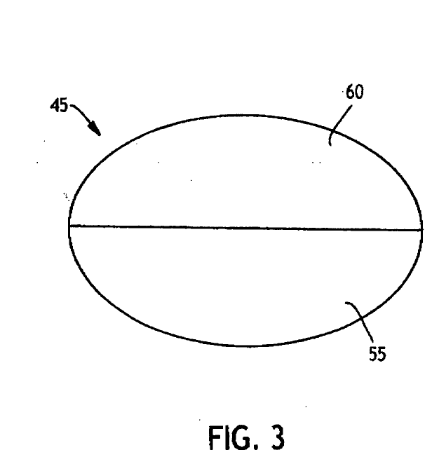

In one aspect of the present invention illustrated in FIG. 3, the dosage form 45 includes an attachment layer 55. When the dosage form 45 is coupled to the tampon body, the attachment layer 55 is positioned adjacent the tampon body 34. The attachment layer 55 may be formed from ingredients specifically chosen to assist in attaching the dosage form 45 to the tampon body 34. An ingredient or ingredients for an attachment layer 55 may be chosen for its/their propensity to melt or react such that the dosage form 45 can be attached to the tampon body 34. The attachment layer ingredient or ingredients may also be chosen for its/their propensity to melt at body temperature, ensuring that the dosage form 45 is separated from the tampon body 34 during use.

In aspects of the present invention, the attachment layer 55 may include excipients, biologically-compatible adhesives, surfactants, and penetration enhancers. An example of a suitable excipient that disperses primarily by melting is a semi-synthetic glyceride; one common family of which is a SUPPOCIRE suppository base, available from GattefosséCorp. SUPPOCIRE suppository base is a semi-synthetic glyceride. The semi-synthetic glyceride used for the attachment layer 55 softens at temperatures around 36° C. and eventually turns to a liquid. At lower temperatures the semi-synthetic glyceride has a waxy/greasy texture similar to butter or lard. When heated to just below its melting temperature, the semi-synthetic glyceride takes on a pasty consistency, allowing it to conform to its surroundings, to mix and attach to adjacent layers of semi-synthetic glycerides, and to mechanically attach to a tampon body 34.

In other aspects of the present invention, the excipient may be any other suitable substance that melts at or near body temperature, including the HYDRO-KOTE line of suppository base fats available from Abitec Corporation. These suppository bases are produced from vegetable fats or vegetable-derived fatty acids, are nontoxic, and are non-reactive toward normal actives. Specific examples include HYDRO-KOTE 102 suppository base made from hydrogenated vegetable oil and HYDRO-KOTE AP-5 suppository base made from partially-hydrogenated vegetable oil. The excipient may also be a cocoa butter suppository base such as a deodorized cocoa butter available from KIC Chemicals, Inc.

In another aspect of the present invention, the excipient may be one that dissolves or disperses in the presence of a liquid such as water or menstrual fluid. One example of a water-soluble excipient is polyethylene glycol (PEG) such as PEG 1450 NF or PEG 400 NF available from J. T. Baker. A second example of a water-soluble excipient is polyethylene oxide (PEO) such as SENTRY Polyox WSR N80 excipient available from The Dow Chemical Company. Another example of a water-soluble excipient is hydroxypropylmethylcellulose (HPMC) such as METHOCEL E15 excipient available from The Dow Chemical Company. Another example of a water-soluble excipient is a glycerin-based excipient such as those available from KIC Chemicals, Inc. Glycerin suppository base includes glycerin and gelatin and is used to treat constipation. Glycerin acts as a lubricant and a mild irritant and stimulates the muscles of the intestine, causing the muscles to contract. Glycerin may also be known as glycerol. Another example of a water-soluble excipient is polyvinyl alcohol (PVA) such as that available from J. T. Baker.

In other aspects of the present invention, the excipient may be a starch-based gel or an impregnated membrane as is known in the art.

An example of a suitable biologically-compatible adhesive is hydroxypropyl methylcellulose (HPMC), available as METHOCEL*K15M from The Dow Chemical Company. An example of a suitable surfactant is polysorbate 80, available from Spectrum Chemical Manufacturing Corp. An example of a suitable penetration enhancer is LABRAFIL M 1944 C nonionic amphiphilic excipient, available from Gattefossé Corp.

The dosage form also includes an active layer 60 positioned away from the tampon body and adjacent to the attachment layer 55, as illustrated in FIG. 3. In aspects of the present invention, the active layer 60 may include excipients, biologically-compatible adhesives, surfactants, and penetration enhancers. Suitable excipients used in the active layer 60 include those described above with reference to the attachment layer 55.

One of the primary purposes of the attachment layer 55 is to inhibit any of the active layer 60 from absorbing into the tampon body 34 during manufacture. A second primary purpose of the attachment layer 55 is to remain at least partially intact longer than the active layer 60 when in use, thus significantly inhibiting the active layer 60 from absorbing into the tampon body 34 during use.

Both purposes are accomplished by providing an attachment layer 55 with properties differentiated from the properties of the active layer 60. For example, the attachment layer 55 and the active layer 60 may be formed from similar or dissimilar materials as long as the attachment layer 55 has a higher melting point than the active layer 60. In another example, the attachment layer 55 and the active layer 60 may be formed from similar or dissimilar materials as long as the attachment layer 55 has a lower solubility than the active layer 60. The properties may also be mixed; an active layer 60 that undergoes a primary structural change through a first mechanism such as melting may be combined with an attachment layer 55 that undergoes a primary structural change through a second mechanism such as dissolution, for example, as long as the attachment layer 55 generally lasts longer in use than the active layer 60.

In a specific example, the active layer 60 is produced primarily from excipient that melts at human body temperature (about 37° C.±). The attachment layer 55 is produced primarily from excipient that melts at a higher temperature (e.g., 42-45° C.). Such a structure would inhibit migration of the therapeutic agent into the tampon body 34 during use because the excipient in the active layer 60 carrying the therapeutic agent melts more quickly than the excipient in the attachment layer 55, which is positioned between the active layer 60 and the tampon body 34. In other words, the excipients are selected such that the attachment layer 55 has a heat of fusion greater than the heat of fusion of the active layer 60. The same effect is found when using an active layer 60 and an attachment layer 55 of differentiated properties, such as different rates of solubility.

In various aspects of the present invention, each of the attachment and active layers 55, 60 of the dosage form 45 disperses by a different mechanism, or by the same mechanism with differentiated properties, to allow at least a portion of the attachment layer 55 to remain in place after the active layer 60 has dispersed. A therapeutic agent is contained in at least the active layer 60, which is generally positioned furthest from the tampon body 34. During the initial minutes of use, the active layer 60 including the therapeutic agent disperses first and the attachment layer 55 closest to the tampon body 34 disperses last. This progressive dispersal of layers forms a shield that minimizes absorption of the therapeutic agent into the tampon body 34. Such a construction allows for robust mechanical attachment of the dosage form 45 to the tampon body 34 and minimizes absorption of the therapeutic agent into the tampon body 34 during manufacture and during the initial minutes of use.

In one aspect of the present invention, each of the attachment and active layers 55, 60 of the dosage form 45 disperses by the same mechanism but with differentiated properties.

For example, the attachment layer 55 may have a higher melt temperature and/or heat of fusion from the active layer 60. The differentiated melt temperatures may be accomplished by forming the attachment and active layers 55, 60 from excipients of different molecular weights. The differentiated melt temperatures may also be accomplished by forming one or both of the attachment and active layers 55, 60 from different mixtures of excipients of different molecular weights. The differentiated melt temperatures may be accomplished by forming one or both of the attachment and active layers 55, 60 from excipients mixed with one or more other components. For example, mixing a SUPPOCIRE suppository base with a Tween 80 surfactant changes the melting point of the suppository base.

In another example, the attachment layer 55 may have a lower dissolution rate from the active layer 60. The differentiated dissolution rates may be accomplished by forming the attachment and active layers 55, 60 from excipients of different molecular weights. The differentiated dissolution rates may also be accomplished by forming one or both of the attachment and active layers 55, 60 from different mixtures of excipients of different molecular weights.

In another aspect of the present invention, each of the attachment and active layers 55, 60 of the dosage form 45 disperses by a different mechanism. For example, one of the attachment and active layers 55, 60 may disperse by melting, and the other of the attachment and active layers 55, 60 may disperse by dissolution.

In one exemplary aspect of the present invention, the attachment layer 55 has a higher melt temperature and/or heat of fusion from the active layer 60. The differentiated melt temperatures may be accomplished by forming the attachment layer 55 from SUPPOCIRE AS2 suppository base and the active layer 60 from SUPPOCIRE CM suppository base. The differentiated melt temperatures may also be accomplished by combinations of various forms of the SUPPOCIRE suppository base and/or different concentrations of the SUPPOCIRE suppository base.

In another exemplary aspect of the present invention, the attachment layer 55 has a lower dissolution rate from the active layer 60. The differentiated dissolution rates may be accomplished by forming the attachment layer 55 from PEG 1450 excipient and the active layer 60 from PEG 400 excipient. The differentiated dissolution rates may also be accomplished by combinations of various forms of PEG and/or different concentrations of PEG. In addition, other excipients may be added as a dispersing agent such as polysorbate or other suitable ingredients that change solubility in water.

In another exemplary aspect of the present invention, one of the attachment and active layers 55, 60 may disperse by melting, and the other of the attachment and active layers 55, 60 may disperse by dissolution. This may also be accomplished by multiple combinations of melting ingredients (e.g., SUPPOCIRE suppository bases) and/or dissolution ingredients (e.g., PEG excipients) with or without other ingredients that alter solubility (e.g., polysorbate).

The dosage form 45 may be provided with additional layers beyond the attachment and active layers 55, 60, including additional active layers. The dosage form 45 may also be formed with more than one therapeutic agent. The additional layers may also include a beneficial agent, an excipient, a carrier component, or some combination of these or other ingredients. If more than one therapeutic agent is provided, the therapeutic agents may be commingled, or may be in separate layers. If more than one therapeutic agent is provided, the therapeutic agents may be arranged such that one therapeutic agent is released in greater quantity before the other therapeutic agent is generally released, in a time-release manner. The dosage form 45 may also be provided with additional layers including one or more ingredients, as dictated by the use of the dosage form 45. To summarize, each layer may contain one or more ingredients, including one or more therapeutic agents, one or more beneficial agents, one or more excipients, one or more carrier components, or a combination of any of these.

In an exemplary aspect of the present invention, a third layer on the top or outside of the dosage form 45 may be used to prepare the vaginal environment for more efficient delivery of a therapeutic agent, which in this case could be in the active or second/middle layer. For example, in a three-layer dosage form 45, the top or outside layer may be used to adjust the pH of the vaginal environment to facilitate absorption of the therapeutic agent. The top or outside layer may include a buffering agent of a low molecular weight PEG base that is highly water soluble and water-miscible at a low temperature; this layer liquefies and changes the vaginal environment to the desired pH. The second or active layer also includes a PEG base but at a higher molecular weight that would take longer to liquefy; this layer also includes the therapeutic agent. Once this layer disperses, the therapeutic agent goes into solution at the degree of ionization optimal for transport across the vaginal mucosa. Finally, the third or attachment layer includes an even higher molecular weight PEG or a SUPPOCIRE suppository base (that is not water dispersible). The PEG material in each layer may be a mix to give the degree of solubility in water and the melt temperature required for each layer.

The term layer as used herein does not solely refer to a stack of vertically-aligned, generally horizontal planar ingredient disks. Layer may refer to any sort of zoning or distribution of ingredients, including vertical slices, concentric shells, asymmetrically-loaded zones, or isolated regions or pockets of ingredients. In other words, layer may refer to any non-homogeneous distribution of ingredients within the dosage form 45.

The dosage form 45 may be shaped and manufactured by any suitable method. In the case of a dosage form having more than one layer, one of the attachment and active layers 55, 60 may be deposited and allowed to cool, dry, or set, as appropriate, before the other of the attachment and active layers 55, 60 is deposited thereon. Succeeding layers may be deposited in the same way until the desired dosage form 45 is created.

The dosage form 45 may be produced by the same manufacturer as the manufacturer of the tampon assembly 40. The dosage form 45 may also be produced by a separate manufacturer and provided to the tampon manufacturer in any suitable manner. As an example, a dosage form manufacturer with a facility specifically designed for pharmaceutical manufacturing that meets current regulatory and quality requirements for drugs and/or devices, as appropriate, may produce the dosage forms 45 under conditions such that homogeneity, concentration, and purity of the dosage form 45 are closely controlled, and such that production is in accordance with applicable regulations. The dosage form 45 may then be sealed and shipped to the manufacturer of the tampon assembly 40. In this manner, the dosage form 45 is produced by a manufacturer with appropriate experience, and the tampon manufacturer may be relieved of establishing a pharmaceutical-production facility. This process is described in more detail in co-pending U.S. patent application Ser. No. 10/335,816 filed on Dec. 31, 2002 and titled “Medicated Tampon.”

The dosage form 45 may be produced by applying the formulation including a therapeutic agent to a substrate as described in co-pending U.S. patent application Ser. No. 11/090,554 filed on Mar. 25, 2005 and titled “Methods of Manufacturing a Medicated Tampon Assembly.”

In one aspect of the present invention, the dosage form 45 is coupled to the tampon body 34 by heating a portion of the dosage form 45 to melt all or some of that portion of the dosage form 45. Such heating may be accomplished using heated air, heated liquid, infrared, or any other suitable heating means. The dosage form 45 is then abutted with the tampon body 34 such that the melted region of the portion is applied with appropriate. pressure to engage the tampon body 34. The melted region of the portion then re-solidifies, becoming attached to the tampon body 34. In one aspect of the present invention, the dosage form 45 is thereby mechanically engaged with the fibers of the tampon body 34. In a different aspect of the present invention, the tampon body 34 may be heated and then put in contact with the dosage form 45, thus using the heat from the tampon body 34 to heat and melt at least part of the dosage form 45.

In a different aspect of the present invention, the dosage form 45 is at least partially coated with a suitable biologically-compatible adhesive such as HPMC or other suitable adhesive and then abutted with the tampon body 34 such that the dosage form 45 is affixed to the tampon body 34. The HPMC may be applied alternatively or additionally to one of the tampon body 34 or the dosage form 45.

In a different aspect of the present invention, usable in either case, the attachment may be accomplished in a manufacturing environment by introducing a small amount of heated, melted excipient such as SUPPOCIRE suppository base or other semi-synthetic glyceride onto the tampon body 34 just prior to introducing the dosage form 45 onto the tampon body 34. The heat contained in the melted excipient partially melts the dosage form 45 and creates a secure bond when both the dosage form 45 and the introduced excipient cool and harden. The heated excipient may be applied alternatively or additionally to one of the attachment layer 55 or the dosage form 45.

In different aspects of the present invention, the dosage form 45 may be coupled to the tampon body 34 by any method described in co-pending U.S. patent application Ser. No. ______ filed on Dec. 7, 2005 and titled “Methods of Attaching a Dosage Form to a Medicated Tampon Assembly,” in co-pending U.S. patent application Ser. No. 11/090,554 filed on Mar. 25, 2005 and titled “Methods of Manufacturing a Medicated Tampon Assembly,” and in co-pending U.S. patent application Ser. No. 11/116,643 filed on Apr. 28, 2005 and titled “Dosage Form Cap for an Applicator.”

In use, and referring to FIG. 2, the medicated tampon assembly 10 functions because the second member 18 is telescopically movable relative to the first member 14. The user may position one or more fingers on the fingergrip ring 28 and one or more fingers on the finger flange 32. The user then squeezes the fingergrip ring 28 and pushes the finger flange 32 toward the fingergrip ring 28 to force the second member 18 further into the first member 14 until the dosage form 45 is expelled from the first member 14. More specifically, as the second member 18 is pushed into the first member 14, the tampon 20 is forced forward against the petals 27. The contact by the tampon 20 causes the petals 27 to radially open to a diameter that is sufficient to allow the tampon 20 to be expelled from the first member 14. With the tampon 20 properly positioned in the vaginal or other cavity, the medicated tampon assembly 10 is withdrawn and properly discarded.

Once the tampon 20 is properly positioned in the vaginal or other cavity, the tampon body 34 absorbs menses and other bodily fluids, and the dosage form 45 delivers the therapeutic agent to the vaginal or other epithelium for local or topical therapeutic action or from there, the therapeutic agent may be transferred to the uterus by normal bodily functions to relieve the condition to be treated.

Aspects of the present invention have been described with reference to various specific and illustrative aspects and techniques. However, it should be understood that many variations and modifications may be made while remaining within the spirit and scope of the invention.

Accordingly, aspects of the present invention are intended to embrace all such alternatives, modifications and variations that fall within the spirit and scope of the appended claims.

We claim:

1. A tampon adapted to deliver a therapeutic agent, the tampon comprising:
a tampon body having a distal end; and
a dosage form affixed to the distal end of the tampon body, the dosage form including
a first active layer dispersible by a first mechanism, the active layer including the therapeutic agent, and
an attachment layer dispersible by a second mechanism, wherein the first mechanism is different from the second mechanism.

2. The tampon of claim 1, wherein the first mechanism is melting.

3. The tampon of claim 1, wherein the second mechanism is dissolution.

4. The tampon of claim 1, wherein the therapeutic agent is selected from the group consisting of nonsteroidal anti-inflammatory drugs (NSAIDs), prostaglandin inhibitors, COX-2 inhibitors, local anesthetics, calcium channel blockers, potassium channel blockers, β-adrenergic agonists, vasodilators, leukotriene blocking agents, smooth muscle inhibitors, and drugs capable of inhibiting dyskinetic muscle contraction.

5. The tampon of claim 4, wherein the NSAID is selected from the group consisting of aspirin, ibuprofen, indomethacin, phenylbutazone, bromfenac, sulindac, nabumetone, ketorolac, mefenamic acid, and naproxen.

6. The tampon of claim 4, wherein the COX-2 inhibitor is selected from the group consisting of celecoxib, meloxicam, rofecoxib, and flosulide.

7. The tampon of claim 4, wherein the local anesthetic is selected from the group consisting of lidocaine, mepivacaine, etidocaine, bupivacaine, 2-chloroprocaine hydrochloride, procaine, and tetracaine hydrochloride.

8. The tampon of claim 4, wherein the calcium channel blocker is selected from the group consisting of diltaizem, israpidine, nimodipine, felodipine, verapamil, nifedipine, nicardipine, and bepridil.

9. The tampon of claim 4, wherein the potassium channel blocker is selected from the group consisting of dofetilide, e-4031, imokalant, sematilide, ambasilide, azimilide, tedisamil, rp58866, sotalol, piroxicam, and ibutilide.

10. The tampon of claim 4, wherein the β-adrenergic agonist is selected from the group consisting of terbutaline, salbutamol, metaproterenol, and ritodrine.

11. The tampon of claim 4, wherein the vasodilator is selected from the group consisting of nitroglycerin, isosorbide dinitrate, and isosorbide mononitrate.

12. The tampon of claim 4, wherein the therapeutic agent is selected from the group consisting of a botanical, a vitamin, calcium, magnesium, a hormone, a prostaglandin synthetase inhibitor, a leukotriene receptor antagonist, an essential fatty acid, a sterol, an anti-inflammatory agent, a chemotherapeutic agent, and an agent to treat infertility.

13. The tampon of claim 12, wherein the botanical is selected from the group consisting of Agnus castus, aloe vera, comfrey, calendula, dong quai, black cohosh, chamomile, evening primrose, Hypericum perforatum, licorice root, black currant seed oil, St. John's wort, tea extracts, lemon balm, capsicum, rosemary, Areca catechu, mung bean, borage seed oil, witch hazel, fenugreek, lavender, soy, Vaccinium extracts, cranberries, blueberries, azaleas (Rhododendron spp.), red onion skin, short and long red bell peppers, Beta vulgaris (beet) root extract, capsanthin, whortleberry, lingenberry, chokeberry, sweet rowan, rowanberry, seabuckhrouberry, crowberry, strawberries, and gooseberries.

14. The tampon of claim 1, wherein the active layer includes an excipient.

15. The tampon of claim 1, wherein the attachment layer includes an excipient.

16. The tampon of claim 1, further comprising a second active layer.

17. The tampon of claim 16, wherein the first active layer and the second active layer are adapted such that the first active layer is substantially dispersed from the dosage form prior to the dispersal of the second active layer.

18. The tampon of claim 1, wherein the dosage form is affixed to the tampon body by partially melting the attachment layer.

19. The tampon of claim 1, wherein the dosage form is affixed to the tampon body using a biologically-compatible adhesive.

20. The tampon of claim 1, wherein the dosage form includes a plurality of therapeutic agents.

21. A dosage form including a therapeutic agent, the dosage form comprising:
a first active layer dispersible by a first mechanism, the first active layer including the therapeutic agent, and
an attachment layer dispersible by a second mechanism, wherein the first mechanism is different from the second mechanism, wherein the first active layer and the attachment layer are adapted such that the first active layer is substantially dispersed from the dosage form prior to the complete dispersal of the attachment layer, and wherein the dosage form is adapted for vaginal delivery.

22. The dosage form of claim 21, wherein the first mechanism is melting.

23. The dosage form of claim 21, wherein the second mechanism is dissolution.

24. The dosage form of claim 21, further comprising a second active layer.

25. The dosage form of claim 24, wherein the first active layer and the second active layer are adapted such that the first active layer is substantially dispersed from the dosage form prior to the dispersal of the second active layer.

26. The dosage form of claim 21, wherein the dosage form includes a plurality of therapeutic agents.

27. The dosage form of claim 21, wherein the dosage form is adapted to be coupled to an absorbent article.
